(19) United States
(12) Patent Application Publication
(10) Pub. No.: US 2005/0131082 A1
(43) Pub. Date: Jun. 16, 2005

(54) Process for reducing the pour point and viscosity of fischer-tropsch wax

(75) Inventors: Stephen E. Henderson, Rodeo, CA (US); David R. Johnson, Petaluma, CA (US); Stephen J. Miller, San Francisco, CA (US); Russell R. Krug, Novato, CA (US)

Correspondence Address: CHEVRON TEXACO CORPORATION, P.O. BOX 6006, SAN RAMON, CA, 94583-0806, US

(73) Assignee: Chevron U.S.A. Inc.

(21) Appl. No.: 10/735,431

(22) Filed: Dec. 12, 2003

(51) Int. Cl. C07C 27/26; C07B 63/02

(52) U.S. Cl. 518/726; 208/106

(57) ABSTRACT

An integrated process for lowering the pour point of Fischer-Tropsch derived wax which comprises (a) collecting separately from a Fischer-Tropsch unit a Fischer-Tropsch wax and a Fischer-Tropsch condensate; (b) pyrolyzing the Fischer-Tropsch wax in a thermal cracking zone under thermal cracking conditions pre-selected to achieve a cracking conversion of the paraffins molecules present in the Fischer-Tropsch wax of at least 10 percent; (c) recovering from the thermal cracking zone a low pour point Fischer-Tropsch derived wax and a Fischer-Tropsch derived overhead product; and (d) mixing at least a portion of the Fischer-Tropsch derived overhead product recovered in step (c) and at least a portion of the Fischer-Tropsch condensate collected in step (a) with at least a portion of the low pour point Fischer-Tropsch derived wax in the proper proportion to produce a Fischer-Tropsch derived waxy product having a pour point equal to or below about 40 degrees C.

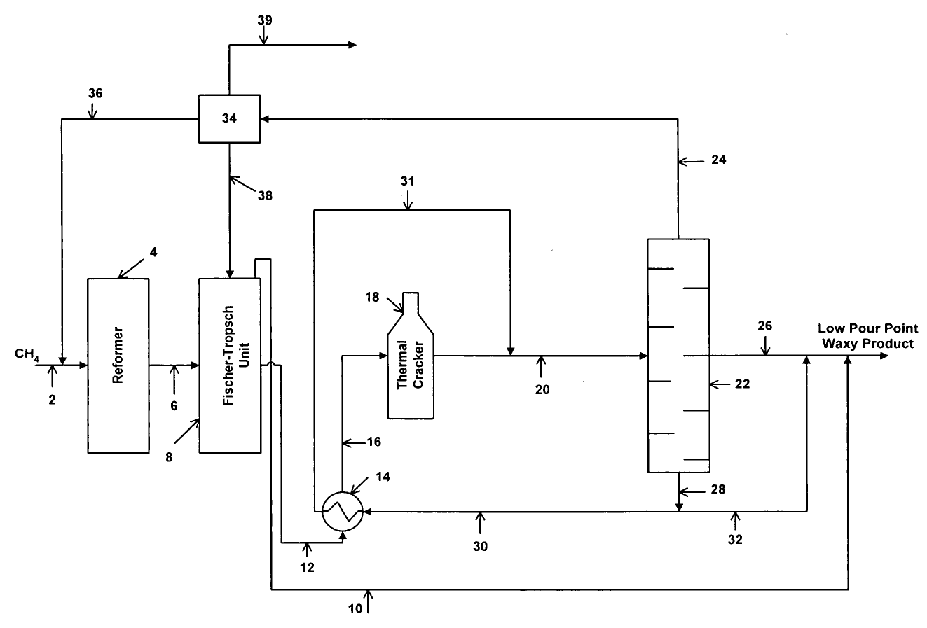

### FIELD OF THE INVENTION

The present invention is directed to a process for lowering the pour point and viscosity of a Fischer-Tropsch wax to facilitate its handling and shipping.

### BACKGROUND OF THE INVENTION

The Fischer-Tropsch process is useful for converting stranded natural gas into higher molecular weight hydrocarbons. In remote locations, there is often no economically attractive way to transport the natural gas produced at the wellhead to market. Previously, stranded natural gas produced in remote oil fields was either re-injected into the well or flared. Neither method of disposing of the natural gas was satisfactory from either an environmental or an economic perspective. However, by operating a Fischer-Tropsch unit at the production site, the methane and normally gaseous hydrocarbons, such as ethane, propane and butane, may be converted into C₅ plus hydrocarbons which may be more readily transported as liquids. Unfortunately, a high molecular weight waxy fraction is also produced which presents its own handling problems.

The hydrocarbons recovered from the Fischer-Tropsch synthesis reactor usually may be classified into three categories based upon a combination of their molecular weight and boiling point. The lowest molecular weight fraction is normally gaseous at ambient temperature and is also the least valuable commercially. Parts of this gaseous fraction may be used locally as fuel, sold as LPG, upgraded by oligomerization to higher molecular weight material, or recycled to the Fischer-Tropsch synthesis unit. The Fischer-Tropsch condensate fraction which usually has a boiling range between about ambient temperature and about 650 degrees F. is normally liquid at ambient temperature and may be readily transported by ship to a refinery where it may serve as a feedstock for upgrading to transportation fuels, such as naphtha, jet and diesel, or used as a feedstock in petrochemical processes, such as ethylene cracking. The Fischer-Tropsch wax fraction is generally a solid at ambient temperature, and, like the condensate, the wax must be further processed in a refinery before it yields commercially valuable products. Unfortunately, the solid wax cannot be pumped at ambient temperatures and consequently is not readily transportable by ship. Therefore, the Fischer-Tropsch wax presents its own handling and transportation problems. See U.S. Pat. No. 6,518,321.

Various methods for processing the Fischer-Tropsch wax prior to transporting it have been proposed. See, for example, U.S. Pat. Nos. 6,268,401 and 6,294,587. Thermal cracking has been proposed for use in lowering the pour point both of conventional petroleum derived waxy crude (U.S. Pat. No. 6,337,011) and of Fischer-Tropsch waxes (PCT Publication WO 99/37737). U.S. Pat. No. 6,379,534 describes a process for lowering the pour point of waxy petroleum derived crude by first separating the waxy crude into its high boiling and low boiling components and then using a combination of thermal cracking of some of the higher boiling hydrocarbons followed by blending back of some lower boiling hydrocarbons to produce a lower pour point crude.

Ideally, a process for preparing the Fischer-Tropsch wax at a remote location prior to shipment will (a) produce a liquid product which is pumpable at mild temperature, (b) use relatively simple equipment, (c) be easy to operate, and (d) require relatively low capital and operating costs. None of the prior processes for handling Fischer-Tropsch wax meet all of these criteria. The process of the present invention does. In addition, the process of the present invention may be integrated with the operation of the Fischer-Tropsch unit to increase the yield of desirable products and reduce the operating expenses.

In paraffinic base residua derived from conventional petroleum, long paraffinic chains attached to aromatic rings are believed to be the primary cause of high pour points and viscosities. Therefore, visbreaking when used with conventional petroleum derived crude is carried out under conditions to optimize the breaking off of these long side chains and their subsequent cracking to shorter molecules with lower viscosities and pour points. See Petroleum Refining: Technology and Economics by James H. Gary and Glenn E. Handwerk (Chapter 5, pages 84-85) 4^{th} Ed., Marcel Dekker N.Y. (2001). Fischer-Tropsch derived hydrocarbons are mainly normal paraffins and, unlike petroleum derived crude, do not contain aromatics. Therefore, it is particularly surprising that mild thermal cracking of Fischer-Tropsch derived materials results in significant large pour point reduction.

As used in this disclosure, the words “comprises” or “comprising” are intended as an open-ended transition meaning the inclusion of the named elements, but not necessarily excluding other unnamed elements. The phrases “consists essentially of” or “consisting essentially of” are intended to mean the exclusion of other elements of any essential significance to the composition. The phrases “consisting of” or “consists of” are intended as a transition meaning the exclusion of all but the recited elements with the exception of only minor traces of impurities.

### SUMMARY OF THE INVENTION

In its broadest aspect, the present invention is directed to a process for lowering the pour point of Fischer-Tropsch derived wax which comprises (a) collecting separately from a Fischer-Tropsch unit a Fischer-Tropsch wax and a Fischer-Tropsch condensate; (b) pyrolyzing the Fischer-Tropsch wax in a thermal cracking zone under thermal cracking conditions pre-selected to achieve a cracking conversion of the paraffin molecules present in the Fischer-Tropsch wax of at least 10 percent; (c) recovering from the thermal cracking zone a thermally cracked Fischer-Tropsch derived wax intermediate having a lower pour point than the Fischer-Tropsch wax; and (d) mixing at least a portion of the Fischer-Tropsch condensate collected in step (a) with at least a portion of the thermally cracked Fischer-Tropsch derived wax intermediate in the proper proportion to produce a Fischer-Tropsch derived waxy product having a pour point equal to or below about 40 degrees C. The present invention is also directed to an integrated process for lowering the pour point of Fischer-Tropsch derived wax which comprises (a) collecting separately from a Fischer-Tropsch unit a Fischer-Tropsch wax and a Fischer-Tropsch condensate; (b) pyrolyzing the Fischer-Tropsch wax in a thermal cracking zone under thermal cracking conditions pre-selected to achieve a cracking conversion of the paraffins molecules present in the Fischer-Tropsch wax of at least 10 percent; (c) recovering from the thermal cracking zone a low pour point Fischer-Tropsch derived wax and a Fischer-Tropsch derived overhead product; and (d) mixing at least a portion of the Fischer-Tropsch derived overhead product recovered in step (c) and at least a portion of the Fischer-Tropsch condensate collected in step (a) with at least a portion of the low pour point Fischer-Tropsch derived wax in the proper proportion to produce a Fischer-Tropsch derived waxy product having a pour point equal to or below about 40 degrees C.

In addition to lowering the pour point of the Fischer-Tropsch derived waxy product, the present invention will also reduce the viscosity.

As will be discussed in greater detail below, in the integrated process, the Fischer-Tropsch derived overhead product recovered from the thermal cracking zone comprises Fischer-Tropsch derived hydrocarbons having a lower boiling range than the low pour point Fischer-Tropsch derived wax. Generally, the Fischer-Tropsch derived overhead product will contain a mixture of C₅ plus hydrocarbons, i.e., hydrocarbons which are normally liquid at ambient temperature, such as, for example, pentane, hexane and heptane; and C₄ minus hydrocarbons, i.e., hydrocarbons which are normally gaseous at ambient temperature, such as, for example, ethane, propane and butane. In addition, depending upon how severe the thermal cracking reactor is operated, the Fischer-Tropsch derived overhead product may also contain a significant amount of methane. Generally, it is advantageous to separate the C₄ minus hydrocarbons from the C₅ plus hydrocarbons. A C₁₋₂ fraction can be isolated and recycled upstream of the syngas generation process, recycled to the Fischer-Tropsch unit, flared, used to produce hydrogen, and/or used for fuel. A C₃₋₄ fraction can be recycled upstream of the syngas generation process, recycled to the Fischer-Tropsch unit, flared, used for fuel, transported in pressurized tankers, and/or transported in refrigerated tankers.

As used in this disclosure, the phrase “Fischer-Tropsch derived” refers to a hydrocarbon stream in which a substantial portion, except for added hydrogen, is derived from a Fischer-Tropsch process regardless of subsequent processing steps. Accordingly, a “Fischer-Tropsch derived liquid waxy product” refers to a highly paraffinic product which comprises a substantial portion of hydrocarbons boiling above about 700 degrees F that was initially derived from the Fischer-Tropsch process.

### BRIEF DESCRIPTION OF THE DRAWING

The FIGURE is a diagram which illustrates an embodiment in which the process of the present invention is integrated with a Fischer-Tropsch unit.

### DETAILED DESCRIPTION OF THE INVENTION

The invention will be more clearly understood by reference to the FIGURE which illustrates an embodiment in which the process of the invention is fully integrated into a Fischer-Tropsch synthesis operation. Methane recovered from the wellhead gas is carried by line 2 to an autothermal reforming unit 4 where the methane is converted into syngas which comprises primarily a mixture of hydrogen and carbon monoxide. The syngas passes from the autothermal reforming unit by line 6 to the Fischer-Tropsch reactor 8. In the Fischer-Tropsch reactor, the syngas is converted into a mixture of hydrocarbons containing anywhere from 1 to 200 plus carbon atoms with the majority falling within the C₅ to C₁₀₀ plus range. As noted previously, the products from the Fischer-Tropsch synthesis may be classified into three categories. One fraction which is normally gaseous at ambient temperature comprises primarily methane and hydrocarbons containing between 2 and about 4 carbon atoms. Although not shown in the Figure, these lower molecular weight hydrocarbons may be recovered. A C₁₂ fraction can be isolated and recycled upstream of the syngas generation process, recycled to the Fischer-Tropsch unit, flared, used to produce hydrogen, and/or used for fuel. A C₃₋₄ fraction can be recycled upstream of the syngas generation process, recycled to the Fischer-Tropsch unit, flared, used for fuel, transported in pressurized tankers, and/or transported in refrigerated tankers. The hydrocarbon fraction containing between about 5 to about 19 carbon atoms is normally liquid at ambient temperature and is referred to in this disclosure as condensate. The condensate is shown in the FIGURE as being collected from the Fischer-Tropsch reactor by line 10. The C₂₀ plus fraction referred to as Fischer-Tropsch wax is shown being collected from the Fischer-Tropsch reactor by line 12 which carries the fraction to a heat exchanger 14 where the temperature of the wax is raised. From the heat exchanger, the Fischer-Tropsch wax is transported by line 16 to the thermal cracking unit 18.

In the thermal cracking unit 18, the Fischer-Tropsch wax is subjected to mild thermal cracking sufficient to significantly reduce the pour point of the wax. The low pour point wax is collected in line 20 which carries it to a fractionation column 22 where any C₄ minus gases are collected as overhead gases in line 24. Hydrocarbons containing from about 5 to about 19 carbon atoms, i.e., normally liquid at ambient temperature, are collected from the fractionation column in line 26. The low pour point wax is shown being collected from the bottom of the fractionation column by line 28 and being split into two streams. One low pour point wax stream is carried by line 30 to heat exchanger 14 where it is used to preheat the Fischer-Tropsch wax going to the thermal cracking unit 18. After passing through the heat exchanger, the cooled low pour point wax stream passes by line 31 back to line 20 to quench the thermal cracking reactions. The second low pour point wax stream is carried by line 32 to be mixed with the C₅ to C₁₉ hydrocarbons in line 26 and the condensate in line 10. The three components are mixed in the proper proportions in line 26 to produce a low pour point waxy product which is liquid at ambient temperature and readily handled by conventional pumping equipment normally available at petroleum loading and unloading facilities. It will be seen that, in this embodiment, the low pour point waxy product formed is a mixture of the low pour point wax, the C₅ to C₁₉ hydrocarbons collected from the thermal cracking unit, and the condensate recovered directly from the Fischer-Tropsch reactor.

Hydrocarbons containing less than 5 carbon atoms are collected from the fractionation column 22 as overhead gases by line 24. The overhead gases are sent to a separator 34 in which the methane is separated from the C₂ to C₅ hydrocarbons. A C₁₋₂ fraction can be recycled upstream of the syngas generation process by line 36, recycled to the Fischer-Tropsch unit by line 38, or, alternatively, via line 39 it may be flared, used to produce hydrogen, and/or used for fuel. A C₃₋₄ fraction can be recycled upstream of the syngas generation process by line 36, recycled to the Fischer-Tropsch unit by line 38, or, alternatively, via line 39 it may be flared, used for fuel, transported in pressurized tankers, and/or transported in refrigerated tankers.

Fischer-Tropsch Process Feedstocks

Natural gas which may be used to generate the synthesis gas used as a feedstock in the Fischer-Tropsch process is an abundant fossil fuel resource. Natural gas is often associated with petroleum production facilities. The composition of natural gas at the wellhead varies, but the major hydrocarbon present is methane. For example, the methane content of natural gas may vary within the range of from about 40 volume percent to 95 volume percent. Other constituents of natural gas may include ethane, propane, butanes, pentane (and heavier hydrocarbons), hydrogen sulfide, carbon dioxide, helium and nitrogen.

Since much of the known reserves for natural gas are found along with crude oil in locations where it not economical to ship the gas to market, the natural gas under such circumstances is often flared or re-injected into the well. In either case, the economic value of the natural gas is lost. In addition, since almost all of the carbon value in the natural gas is converted into products by the Fischer-Tropsch process, minimal carbon dioxide is released into the atmosphere.

Natural gas is classified as dry or wet depending upon the amount of condensable hydrocarbons contained in it. Condensable hydrocarbons generally comprise C₃ plus hydrocarbons although some ethane may be included. Gas conditioning is required to alter the composition of wellhead gas, processing facilities usually being located in or near the production fields. Conventional processing of wellhead natural gas yields processed natural gas containing at least a major amount of methane.

Typically, synthesis gas contains hydrogen and carbon monoxide, and may include minor amounts of carbon dioxide and/or water. The presence of certain contaminants, such as sulfur, nitrogen, halogen, selenium, phosphorus and arsenic contaminants, in the syngas are undesirable. For this reason, it is preferred to remove sulfur and other contaminants from the feed before performing the Fischer-Tropsch chemistry. Means for removing these contaminants are well known to those of skill in the art. For example, ZnO guardbeds are preferred for removing sulfur impurities. Means for removing other contaminants are well known to those of skill in the art.

It is also possible to use methane derived from other sources in the Fischer-Tropsch process. Methane can be derived from a variety of other sources, such as the fuel gas system, the gasification of the heavy carbonaceous materials such as may be found in coal, coker bottoms, and residuum, or even the reduction of methanol.

The synthesis gas used to carry out the present invention can be generated using steam methane reforming, partial oxidation or gasification, or a combined reforming or autothermal reforming process.

Fischer-Tropsch Synthesis

In the Fischer-Tropsch synthesis process, liquid and gaseous hydrocarbons are formed by contacting a synthesis gas (syngas) comprising a mixture of hydrogen and carbon monoxide with a Fischer-Tropsch catalyst under suitable temperature and pressure reactive conditions. The Fischer-Tropsch reaction is typically conducted at temperatures of from about 300 degrees to about 700 degrees F. (149 degrees to 371 degrees C.), preferably from about 400 degrees to about 550 degrees F. (204 degrees to 228 degrees C.); pressures of from about 10 to about 600 psia (0.7 to 41 bars), preferably 30 to 300 psia (2 to 21 bars); and catalyst space velocities of from about 100 to about 10,000 cc/g/hr., preferably 300 to 3,000 cc/g/hr.

The products may range from C₁ to C₂₀₀ plus hydrocarbons with a majority in the C₅to C₁₀₀ plus range. The reaction can be conducted in a variety of reactor types, for example, fixed bed reactors containing one or more catalyst beds, slurry reactors, fluidized bed reactors, or a combination of different type reactors. Such reaction processes and reactors are well known and documented in the literature. Slurry Fischer-Tropsch processes, which is a preferred process in the practice of the invention, utilize superior heat (and mass) transfer characteristics for the strongly exothermic synthesis reaction and are able to produce relatively high molecular weight, paraffinic hydrocarbons when using a cobalt catalyst. In a slurry process, a syngas comprising a mixture of hydrogen and carbon monoxide is bubbled up as a third phase through a slurry in a reactor which comprises a particulate Fischer-Tropsch type hydrocarbon synthesis catalyst dispersed and suspended in a slurry liquid comprising hydrocarbon products of the synthesis reaction which are liquid at the reaction conditions. The mole ratio of the hydrogen to the carbon monoxide may broadly range from about 0.5 to about 4, but is more typically within the range of from about 0.7 to about 2.75 and preferably from about 0.7 to about 2.5. A particularly preferred Fischer-Tropsch process is taught in EP0609079, also completely incorporated herein by reference for all purposes.

Suitable Fischer-Tropsch catalysts comprise one or more Group VIII catalytic metals such as Fe, Ni, Co, Ru and Re, with cobalt being preferred. Additionally, a suitable catalyst may contain a promoter. Thus, a preferred Fischer-Tropsch catalyst comprises effective amounts of cobalt and one or more of Re, Ru, Pt, Fe, Ni, Th, Zr, Hf, U, Mg and La on a suitable inorganic support material, preferably one which comprises one or more refractory metal oxides. In general, the amount of cobalt present in the catalyst is between about 1 and about 50 weight percent of the total catalyst composition. The catalysts can also contain basic oxide promoters such as ThO₂, La₂O₃, MgO, and TiO₂, promoters such as ZrO₂, noble metals (Pt, Pd, Ru, Rh, Os, Ir), coinage metals (Cu, Ag, Au), and other transition metals such as Fe, Mn, Ni, and Re. Suitable support materials include alumina, silica, magnesia and titania or mixtures thereof. Preferred supports for cobalt containing catalysts comprise alumina or titania. Useful catalysts and their preparation are known and illustrated in U.S. Pat. No. 4,568,663, which is intended to be illustrative but non-limiting relative to catalyst selection.

The products as they are recovered from the Fischer-Tropsch operation may be divided into three fractions, a gaseous fraction consisting of very light products, a condensate fraction generally boiling in the range of naphtha and diesel, and a high boiling Fischer-Tropsch wax fraction which is normally solid at ambient temperatures. In the present invention, the wax fraction is recovered separately from the condensate/light product fraction and sent to the thermal cracking unit. The condensate fraction is preferably separated from the light product fraction prior to being blended back into the low pour point wax product recovered from the thermal cracker. The light fraction may be recycled to the Fischer-Tropsch reactor, used to fuel furnaces within the facility, sold as heating fuel, or flared. If sufficient methane is present in the light fraction to justify its separation from the C₂ to C₄ hydrocarbons, it may be recycled to the reformer for conversion into syngas.

Thermal Cracking

The thermal cracking step employed in the process of the present invention is intended to lower the pour point of the Fischer-Tropsch wax by cracking the paraffin molecules into lower molecular weight olefins. At the same time, the viscosity is also reduced. Although batch pyrolysis reactors such as employed in delayed coking or in cyclic batch operations could be used to carry out this step, generally a continuous flow-through operation is preferred in which the feed is first preheated to a temperature sufficient to vaporize most or all of the feed after which the vapor is passed through a tube or tubes. A desirable option is to bleed any remaining nonvaporized hydrocarbons prior to entering the tubes in the cracking furnace. Preferably, the thermal cracking is conducted in the presence of steam which serves as a heat source and also helps suppress coking in the reactor. Details of a typical steam thermal cracking process may be found in U.S. Pat. No. 4,042,488, hereby incorporated by reference in its entirety. Although catalyst is generally not used in carrying out the thermal cracking operation, it is possible to conduct the operation in a fluidized bed in which the vaporized feed is contacted with hot fluidized inert particles, such as fluidized particles of coke.

In the pyrolysis zone, the cracking conditions should be sufficient to provide a cracking conversion of at least 10 percent by weight of the paraffins present. Preferably, the cracking conversion will be at least 20 percent by weight, more preferably at least 30 percent by weight, and most preferably at least 50 percent by weight. The term “cracking conversion” relates to the percentage of the feed boiling above a reference temperature (e.g., the initial boiling point) which is converted to products boiling below the reference temperature. The optimal temperature and other conditions in the pyrolysis zone for the cracking operation will vary somewhat depending on the feed. In general, the temperature must be high enough to maintain the feed in the vapor phase but not so high that the feed is overcracked, i.e., the temperature and conditions should not be so severe that excessive C₄ minus hydrocarbons are generated. The temperature in the pyrolysis zone normally will be maintained at a temperature of between about 800 degrees F. (425 degrees C.) and about 950 degrees F. (510 degrees C.). The optimal temperature range for the pyrolysis zone will depend upon the endpoint of the feed. In general, the higher the carbon number, the higher the temperature required to achieve sufficient conversion to lower the pour point to an acceptable level. Accordingly, some routine experimentation may be necessary to identify the optimal cracking conditions for a specific feed. The pyrolysis zone usually will employ pressures maintained between about 0 atmospheres and about 5 atmospheres, with pressures in the range of from about 0 to about 2 atmospheres generally being preferred. Although the optimal residence time of the wax fraction in the reactor will vary depending on the temperature and pressure in the pyrolysis zone, typical residence times are generally in the range of from about 1.5 seconds to about 500 seconds, with the preferred range being between about 5 seconds and about 300 seconds.

In carrying out the process of the present invention, it is preferred that the Fischer-Tropsch derived wax intermediate recovered from the thermal cracking zone has a pour point of less than about 45 degrees F.

Fischer-Tropsch Derived Waxy Product

In its simplest embodiment, the Fischer-Tropsch derived waxy product is a blend of the Fischer-Tropsch derived wax intermediate recovered from the thermal cracking zone and the condensate recovered directly from the Fischer-Tropsch reactor. The Fischer-Tropsch derived waxy product should have a pour point below about 40 degrees C. and preferably will have a pour point below about 20 degrees C. As already noted, the Fischer-Tropsch derived waxy product usually will have a significantly reduced viscosity as compared to the uncracked Fischer-Tropsch wax. In addition to the condensate, the blend usually will also contain C₅ to about C₁₉ hydrocarbons which are formed in the thermal cracking unit due to the cracking of the wax molecules. The various components are blended in the proper proportion to provide a product which may be pumped at ambient temperature and that will remain liquid during transportation. One skilled in the art will recognize that the proportion of each of the components will vary depending on such factors as the desired pour point of the Fischer-Tropsch derived waxy product, the pour point of the Fischer-Tropsch derived wax intermediate, the pour point of the condensate and the C₅ to about C₁₉ hydrocarbons, and the ambient temperature. Obviously, a pour point suitable for producing a pumpable Fischer-Tropsch derived waxy product in the tropics may not be satisfactory to produce a pumpable blend in the arctic. In order to lower the pour point, it may be necessary to increase the cracking conversion in the thermal cracking unit and/or increase the proportion of the lighter molecular weight hydrocarbons in the blend, i.e., the condensate and C₅ to about C₁₉ hydrocarbons.

The Fischer-Tropsch waxy product may also contain other materials so long as they do not raise the pour point above an acceptable level. For example, a conventional petroleum derived crude having a moderate pour point may be blended with the Fischer-Tropsch waxy product if so desired. Since Fischer-Tropsch units are often located in or near petroleum production facilities, it may be desirable to transport a blend containing both the Fischer-Tropsch waxy product and conventional crude.

The following examples are intended to illustrate the invention, but are not intended to be interpreted as limitations on the invention.

### EXAMPLES

### Example 1

The thermal cracking pilot plant used in the following examples employed a 42 inch long tubular reactor, 0.75-inch OD, 0.56-inch ID containing 175 cc of 12 mesh alundum. A commercial FT wax, C80 from Moore and Munger, Inc. (Two Corporate Drive, Suite 434, Shelton, Conn. 06484) having a pour point of 82 degrees C. and a viscosity at 100 degrees C. of 8.445 cSt, was fed to the reactor upflow at 2 LHSV, based on the alundum volume. Nitrogen gas was also fed to the reactor at a rate of 500 SCF N₂/bbl wax feed. The total pressure was 200 psig. The reactor temperature was 850 degrees F. (454 degrees C.). The reactor effluent was stripped to remove C₄ minus gases, and then distilled into C₅ to 650 degrees F. and 650 degrees F. plus fractions.

Blending the 650 degrees F. minus and 650 degrees F. plus fractions recovered from the pilot plant yielded a whole thermal cracked C₅ plus product with a pour point of 39 degrees C. and a viscosity at 100 degrees C. of 1.79 cSt. It will be noted that the thermal cracked C₅ plus product also had a significantly reduced viscosity as compared to the original C80 FT wax.

Blending 50 weight percent of this C₅ plus thermal cracked product with 50 weight percent of a Fischer-Tropsch condensate having boiling range as shown in Table 1 yielded a C₅ plus product with a pour point of 13 degrees C. (Blend No. 2 in Table 2).

TABLE 1
FT Condensate Properties

| API Gravity | 56.6 |
|---|---|
| D2287 Simulated Distillation | ° F. |
| 0.5 wt% | 76 |
| 5 wt % | 193 |
| 10 wt % | 243 |
| 30 wt % | 339 |
| 50 wt % | 415 |
| 70 wt % | 494 |
| 90 wt % | 569 |
| 95 wt % | 595 |
| 99.5 wt % | 661 |

### Example 2

A blend of 50 weight percent C80 Fischer-Tropsch wax (pour point 82 degrees C.) plus 50 weight percent Arabian Medium crude oil (pour point −35 degrees C.), identified as Blend No. 3 in Table 2, had a pour point of 62 degrees C. which is generally too high to be transported by conventional means. A blend of 25 weight percent Fischer-Tropsch wax, 25 weight percent Fischer-Tropsch condensate, and 50 weight percent Arabian Medium crude (Blend No. 4 in Table 2) had a pour point of 53 degrees C. which was still too high to be transported by conventional means.

### Example 3

Another blend containing 25 weight percent thermal cracked wax, 25 weight percent Fischer-Tropsch condensate, and 50 weight percent Arabian Medium crude (Blend No. 5 in Table 2) was found to have a pour point of 9 degrees C.

This illustrates that blends within the scope of the invention may also include a conventional petroleum derived crude having a moderate pour point. Table 2 summarizes the pour points of these blends described in the above examples:

TABLE 2

| | Wt % in Each Blend | | | | |
|---|---|---|---|---|---|
| | FT | | | Arabian | |
| | Wax | FT | | Medium | |
| Blend | C80 | Condensate | TC Wax | Crude | Pour Pt, ° C. |
| | 100 | | | | 82 |
| | | 100 | | | −13 |
| | | | 100 | | 39 |
| | | | | 100 | −35 |
| 1 | 50 | 50 | | | 62 |
| 2 | | 50 | 50 | | 13 |
| 3 | 50 | | | 50 | 62 |
| 4 | 25 | 25 | | 50 | 53 |
| 5 | | 25 | 25 | 50 | 9 |

1. An integrated process for lowering the pour point of Fischer-Tropsch derived wax which comprises:
(a) collecting separately from a Fischer-Tropsch unit a Fischer-Tropsch wax and a Fischer-Tropsch condensate;
(b) pyrolyzing the Fischer-Tropsch wax in a thermal cracking zone under thermal cracking conditions pre-selected to achieve a cracking conversion of the paraffins molecules present in the Fischer-Tropsch wax of at least 10 percent;
(c) recovering from the thermal cracking zone a low pour point Fischer-Tropsch derived wax and a Fischer-Tropsch derived overhead product; and
(d) mixing at least a portion of the Fischer-Tropsch derived overhead product recovered in step (c) and at least a portion of the Fischer-Tropsch condensate collected in step (a) with at least a portion of the low pour point Fischer-Tropsch derived wax in the proper proportion to produce a Fischer-Tropsch derived waxy product having a pour point equal to or below about 40 degrees C.

2. The process of claim 1 wherein the thermal cracking conditions in the thermal cracking zone are pre-selected to achieve a cracking conversion of at least 20 percent.

3. The process of claim 2 wherein the thermal cracking conditions in the thermal cracking zone are pre-selected to achieve a cracking conversion of at least 30 percent.

4. The process of claim 3 wherein the thermal cracking conditions in the thermal cracking zone are pre-selected to achieve a cracking conversion of at least 50 percent.

5. The process of claim 1 wherein the Fischer-Tropsch derived waxy product of step (d) has a pour point below about 20 degrees C.

6. The process of claim 1 wherein the Fischer-Tropsch derived overhead product of step (c) is further separated prior to step (d) into a C₅ plus hydrocarbon product and a C₄ minus hydrocarbon product and the C₅ plus hydrocarbon product is mixed with the Fischer-Tropsch condensate and the low pour point Fischer-Tropsch derived wax in step (d) to produce the Fischer-Tropsch derived waxy product.

7. The process of claim 6 wherein the C₄ minus hydrocarbon product is recycled to the Fischer-Tropsch unit.

8. The process of claim 6 wherein methane is separately recovered from the C₄ minus hydrocarbon product prior to the C₄ minus hydrocarbon product being recycled to the Fischer-Tropsch unit and the methane is recycled to a reformer for conversion into syngas for use as feed to the Fischer-Tropsch unit.

9. The process of claim 1 further including the step of blending with the Fischer-Tropsch waxy product a petroleum derived crude.

10. The process of claim 1 wherein the Fischer-Tropsch derived waxy product also has a reduced viscosity as compared to the Fischer-Tropsch wax.

11. A process for lowering the pour point of Fischer-Tropsch derived wax which comprises:
(a) collecting separately from a Fischer-Tropsch unit a Fischer-Tropsch wax and a Fischer-Tropsch condensate;
(b) pyrolyzing the Fischer-Tropsch wax in a thermal cracking zone under thermal cracking conditions pre-selected to achieve a cracking conversion of the paraffins molecules present in the Fischer-Tropsch wax of at least 10 percent;
(c) recovering from the thermal cracking zone a thermally cracked Fischer-Tropsch derived wax intermediate having a lower pour point than the Fischer-Tropsch wax; and
(d) mixing at least a portion of the Fischer-Tropsch condensate collected in step (a) with at least a portion of the thermally cracked Fischer-Tropsch derived wax intermediate in the proper proportion to produce a Fischer-Tropsch derived waxy product having a pour point equal to or below about 40 degrees C.

12. The process of claim 11 wherein the thermal cracking conditions in the thermal cracking zone are pre-selected to achieve a cracking conversion of at least 20 percent.

13. The process of claim 12 wherein the thermal cracking conditions in the thermal cracking zone are pre-selected to achieve a cracking conversion of at least 30 percent.

14. The process of claim 13 wherein the thermal cracking conditions in the thermal cracking zone are pre-selected to achieve a cracking conversion of at least 50 percent.

15. The process of claim 11 wherein the thermally cracked Fischer-Tropsch derived wax intermediate has a pour point of less than about 45 degrees C.

16. The process of claim 11 wherein the Fischer-Tropsch derived waxy product of step (d) has a pour point below about 20 degrees C.

17. The process of claim 11 further including the step of blending with the Fischer-Tropsch waxy product a petroleum derived crude.

18. The process of claim 11 wherein the Fischer-Tropsch derived waxy product also has a reduced viscosity as compared to the Fischer-Tropsch wax.
